(19) United States
(12) Patent Application Publication
(10) Pub. No.: US 2007/0048366 A1
(43) Pub. Date: Mar. 1, 2007

(54) Gelatin-based coatings having improved durability

(76) Inventors: Jen-Chi Chen, Morrisville, PA (US); Joseph Luber, Quakertown, PA (US); Frank J. Bunick, Randolph, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/212,956

(22) Filed: Aug. 26, 2005

(51) Int. Cl. A61K 9/64; A61K 38/39

(52) U.S. Cl. 424/456; 514/2

(57) ABSTRACT

The present invention relates to a process for manufacturing gelatin products that have improved stability, particularly as to the dissolution rates and/or reduced degree of crosslinking and the gelatin formulations per se. A further aspect of the invention is use of the improved gelatin compositions for use as a coating for dosage forms or dosage form inserts.

### FIELD OF THE INVENTION

This invention relates to a process for the enhancement of the stability of gelatin products against higher storage temperatures, humidity and/or chemically influenced crosslinking, as well as suitable gelatin compositions and their use as a coating for tablets or as a dosage form insert.

### DESCRIPTION OF THE PRIOR ART

Gelatin is widely used in the pharmaceutical industry as well as in the health food supplement market to manufacture capsules as containers or as coating agents for the dosage forms, or as adjuvants or excipients in pharmaceutical preparations like tablets. A primary objective of these dosage forms is to have a good disintegration after being administrated in order to enable a fast dissolution of the active substances in the appropriate digestive organ. Any delay of the disintegration could retard or even reduce the effect of the drug. It is important for the selected disintegration characteristic of a particular dosage form remain stable over time when finished products are stored prior to use. Extensive dissolution stability testing has been conducted to assess this stability.

Unfortunately, as has been widely described in the literature, it is known that gelatin coated products can exhibit a delay in disintegration over time. One possible cause of the problem could be exposure to certain aldehydes at the initial stage or originating from the decomposition of the drug or one of the excipients over time. The mechanism of this chemical interaction named “crosslinking” has been well understood as action of the aldehyde on the free amino groups of the amino acids and especially the lysine and arginine. It has also been used further in the sense that overcrosslinking of the gelatin would make it totally insoluble and inappropriate for a dosage form intended for the immediate release of the active ingredient.

Various patents have been published which deal with this issue. The resistance to crosslinking by formaldehyde can be obtained by chemically modifying the gelatin (succinylated gelatin), by adding ions in it or silicones or peptides. Another procedure to protect gelatin from crosslinking is to include in the formulation of the drug a formaldehyde scavenger.

### SUMMARY OF THE INVENTION

The present invention relates to a process for manufacturing gelatin products that have improved stability, particularly as to the dissolution rates and/or reduced degree of crosslinking and the gelatin formulations per se. A further aspect of the invention is use of the improved gelatin compositions for use as a coating for dosage forms or dosage form inserts.

### DETAILED DESCRIPTION OF PREFERRED EMBODIMENTS

In one embodiment of the invention, the gelatin formulation contains a natural, thermogelling polymer. Such materials are commonly a tasteless and colorless mixture of derived proteins of the albuminous class that is ordinarily soluble in warm water. Two types of gelatin—Type A and Type B—are used. Type A gelatin is a derivative of acid-treated raw materials. Type B gelatin is a derivative of alkali-treated raw materials. The moisture content of gelatin, as well as its Bloom strength, composition and original gelatin processing conditions, determine its transition temperature between liquid and solid. Bloom is a standard measure of the strength of a gelatin gel, and is roughly correlated with molecular weight. Bloom is defined as the weight in grams required to move a half-inch diameter plastic plunger 4 mm into a 6.67% gelatin gel that has been held at 10° C. for 17 hours. In a preferred embodiment, the flowable material is an aqueous solution comprising 20 to 40 percent 275 Bloom pork skin gelatin, and approximately 60 to 80% water.

The improved gelatin formulations include one or more stabilization additives. The stabilization additives are selected from the group consisting of inorganic acids and their conjugate bases, their associated metal salts and mixtures thereof. Examples of suitable inorganic acids are: phosphoric acid, boric acid, hydrochloric acid, and sulfuric acid. Inorganic acids do not include any carbon atoms in their molecular structure. Examples of suitable metal salts of inorganic acids include but are not limited to sodium phosphate monobasic, potassium phosphate monobasic, sodium bisulfate and potassium bisulfate.

The amount of stabilization additives is up to about 4%, preferably 0.1 to about 2% by weight of the dry gelatin. Each stabilization additive can be added alone or in combination with one or more stabilizing additives having anti-crosslinking properties.

The modified gelatin formulations are particularly useful for coating dosage forms. As used herein, the term “dosage form” applies to any solid object, semi-solid, or liquid composition designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the gastro-intestinal tract of a human.

The core may be any solid form. The core may be prepared by any suitable method, including for example compression or molding. As used herein, “core” refers to a material that is at least partially enveloped or surrounded by another material. Preferably, the core is a self-contained unitary object, such as a tablet or capsule. Typically, the core comprises a solid, for example, the core may be a compressed or molded tablet, hard or soft capsule, suppository, or a confectionery form such as a lozenge, nougat, caramel, fondant, or fat based composition. In certain other embodiments, the core or a portion thereof may be in the form of a semi-solid or a liquid in the finished dosage form. For example the core may comprise a liquid filled capsule, or a semisolid fondant material. In embodiments in which the core comprises a flowable component, such as a plurality of granules or particles, or a liquid, the core preferably additionally comprises an enveloping component, such as a capsule shell, or a coating, for containing the flowable material. In certain particular embodiments in which the core comprises an enveloping component, the shell or shell portions of the present invention are in direct contact with the enveloping component of the core, which separates the shell from the flowable component of the core.

Suitable active ingredients for use in this invention include for example pharmaceuticals, minerals, vitamins and other nutraceuticals; oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the dosage form comprises at least about 1 weight percent, preferably, the dosage form comprises at least about 5 weight percent, e.g. at least about 25 weight percent of a combination of one or more active ingredients. In one preferred embodiment, a core comprises a total of at least about 50 weight percent, e.g. at least about 70 weight percent, say at least about 80 weight percent (based on the weight of the core) of one or more active ingredients. In one embodiment the core is a compressed tablet having a hardness from about 2 to about 30 kp/cm², e.g. from about 6 to about 25 kp/cm². “Hardness” is a term used in the art to describe the diametric breaking strength of either the core or the coated solid dosage form as measured by conventional pharmaceutical hardness testing equipment, such as a Schleuniger Hardness Tester. In order to compare values across different size tablets, the breaking strength must be normalized for the area of the break. This normalized value, expressed in kp/cm², is sometimes referred in the art as tablet tensile strength.

The dosage form can preferably include a compressed core. Suitable shapes for compressed cores include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference) (the tablet shape corresponds inversely to the shape of the compression tooling). The core may then be applied by any suitable method, including molding, dipping, enrobing, compression coating, or spray coating with a shell containing the modified gelatin coating. In certain embodiments the shell may be formed over a barrier layer, while in others the shell may be formed first, and a barrier layer added to the shell. Optionally, one or more intermediate film, i.e. “subcoating” layers can be applied over the barrier layer or core and under the shell coating.

The core typically comprises active ingredient and a variety of excipients, depending on the method by which it is made. In embodiments in which the core is made by compression, suitable excipients include fillers, binders, disintegrants, lubricants, glidants, and the like, as known in the art. In embodiments in which the core is made by compression and additionally confers modified release of an active ingredient contained therein, such core preferably further comprises a release-modifying compressible excipient.

Suitable fillers for use in making the core by compression include water-soluble compressible carbohydrates such as sugars, which include dextrose, sucrose, maltose, and lactose, sugar-alcohols, which include mannitol, sorbitol, maltitol, xylitol, starch hydrolysates, which include dextrins, and maltodextrins, and the like, water insoluble plastically deforming materials such as microcrystalline cellulose or other cellulosic derivatives, water-insoluble brittle fracture materials such as dicalcium phosphate, tricalcium phosphate and the like and mixtures thereof.

Suitable binders for making the core by compression include dry binders such as polyvinyl pyrrolidone, hydroxypropylmethylcellulose, hydroxypropylcellulose and the like; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, starches, and the like; and derivatives and mixtures thereof.

Suitable disintegrants for making the core by compression, include sodium starch glycolate, cross-linked polyvinylpyrrolidone, cross-linked carboxymethylcellulose, starches, microcrystalline cellulose, and the like. Suitable lubricants for making the core by compression include long chain fatty acids and their salts, such as magnesium stearate and stearic acid, talc, glycerides and waxes. Suitable glidants for making the core by compression, include colloidal silicon dioxide, and the like.

In embodiments wherein one or more cores are prepared by compression, a dry blending (i.e. direct compression), or wet granulation process may be employed, as known in the art. In a dry blending (direct compression) method, the active ingredient or ingredients, together with the excipients, are blended in a suitable blender, than transferred directly to a compression machine for pressing into tablets. In a wet granulation method, the active ingredient or ingredients, appropriate excipients, and a solution or dispersion of a wet binder (e.g. an aqueous cooked starch paste, or solution of polyvinyl pyrrolidone) are mixed and granulated. Alternatively a dry binder may be included among the excipients, and the mixture may be granulated with water or other suitable solvent. Suitable apparatuses for wet granulation are known in the art, including low shear, e.g. planetary mixers; high shear mixers; and fluid beds, including rotary fluid beds. The resulting granulated material is dried, and optionally dry-blended with further ingredients, e.g. adjuvants and/or excipients such as for example lubricants, colorants, and the like. The final dry blend is then suitable for compression. Methods for direct compression and wet granulation processes are known in the art, and are described in detail in, for example, Lachman, et al., The Theory and Practice of Industrial Pharmacy, Chapter 11 (3rd ed. 1986).

The dry-blended, or wet granulated, powder mixture is typically compacted into tablets using a rotary compression machine as known in the art, such as for example those commercially available from Fette America Inc., Rockaway, N.J., or Manesty Machines LTD, Liverpool, UK. In a rotary compression machine, a metered volume of powder is filled into a die cavity, which rotates as part of a “die table” from the filling position to a compaction position where the powder is compacted between an upper and a lower punch to an ejection position where the resulting tablet is pushed from the die cavity by the lower punch and guided to an ejection chute by a stationary “take-off” bar.

In one optional embodiment, the core may be prepared by the compression methods and apparatus described in copending U.S. Pat. No. 6,767,200, the disclosure of which is incorporated herein by reference. Specifically, the core is made using a rotary compression module comprising a fill zone, insertion zone, compression zone, ejection zone, and purge zone in a single apparatus having a double row die construction as shown in FIG. 6 of U.S. Pat. No. 6,767,200. The dies of the compression module are preferably filled using the assistance of a vacuum, with filters located in or near each die.

Cores made by compression may be single or multi-layer, for example bi-layer, tablets. The cores have a density of at least about 0.9 g/cc, e.g. at least about 1.0 g/cc and a percent porosity of less than 40%, preferably less than 35%, most preferably 30%. Porosity of a powder is a ratio of void volume to bulk volume. The void volume is the volume of spaces between the particles, while the bulk volume is the total, space occupied. Percent porosity is that ratio expressed as a percentage. These values can be measured using a mercury intrusion porosimeter, such as the Autopore IV 9500 V1.05, available from Micrometics Corporation, at a mercury filling pressure of 1.32 to 1.33 psia, mercury contact angle of 130 degrees, and surface tension 485 dynes/cm. Exemplary cores include a 385 mg compressed soft tablet with a volume of 0.4 cubic centimeters, and a 586 mg compressed tablet with a volume of about 0.5 cc.

The gelatin containing shell may be applied via several methods known in the art including molding, dipping, and enrobing. The gelatin-containing shell is preferably provided on dosage form in a two stage injection molding process, such as the injection molding processes described in detail in published US Patent Application 2005/0074514 or in U.S. Patent Application 2003-0124183 A1, published Jul. 3, 2003, which are incorporated herein by reference.

In these embodiments, a core or shell is formed by injecting a flowable material into a molding chamber. The flowable material comprises a gelatin containing material at a temperature above its melting point but below the decomposition temperature of any active ingredient contained therein. The starting material cools and solidifies in the molding chamber into a shaped form (i.e., having the shape of the mold). The flowable material may comprise solid particles suspended in a molten matrix, for example a gelatin-containing polymer matrix. The flowable material may be completely molten or in the form of a paste. The flowable material may comprise an active ingredient dissolved in a molten material. The flowable material may comprise solid particles dispersed in a fluid carrier.

In the thermal cycle molding method and apparatus of published U.S. patent application US-2003-0086973 a thermal cycle molding module having the general configuration shown therein. The thermal cycle molding module 200 comprises a rotor 202 around which a plurality of mold units 204 are disposed. The thermal cycle molding module includes a reservoir 206 for holding flowable material. In addition, the thermal cycle molding module is provided with a temperature control system for rapidly heating and cooling the mold units.

The mold units may comprise center mold assemblies, upper mold assemblies, and lower mold assemblies which mate to form mold cavities having a desired shape, for instance of a core or a shell surrounding one or more cores. As the rotor rotates, opposing center and upper mold assemblies or opposing center and lower mold assemblies close. Flowable material, which is heated to a flowable state in reservoir, is injected into the resulting mold cavities. The temperature of the flowable material is then decreased, hardening the flowable material. The mold assemblies open and eject the finished product. Shell coating is performed in two steps, each half of the dosage forms being coated separately via rotation of the center mold assembly.

In one embodiment, the compression module of copending U.S. Pat. No. 6,767,200 may be employed to make the core and the shell is applied to the core using a thermal cycle or zero cycle molding module as described above. A transfer device as described in U.S. Pat. No. 6,742,646, the disclosure of which is incorporated herein by reference, may be used to transfer the cores from the compression module to the thermal cycle molding module. The transfer module preferably comprises a plurality of transfer units attached in cantilever fashion to a belt. The transfer device rotates and operates in sync with the compression module and the thermal cycle molding module to which it is coupled. Transfer units comprise retainers for holding cores as they travel around the transfer device.

In one embodiment, tablets or hard capsules may be coated with the gelatin composition of the present invention via known gelatin-dipping process parameters and equipment. Details of such equipment and processing conditions are known in the art and are disclosed at, for example, U.S. Pat. No. 4,820,524, which is incorporated by reference herein.

Another method of applying the gelatin coating composition of the present invention to a core is via an enrobing process wherein two separate films made of gelatinous material are applied to opposite sides of a core by a pair of rotary dies. A detailed description of this process is provided, for example, in U.S. Pat. Nos. 5,146,730 and 5,459,983, and the entire contents and disclosures of both of these patents are hereby incorporated herein by reference.

In one embodiment, the improved gelatin formulations described herein are used to produce dosage forms having gelatin-containing coatings. The gelatin-containing coatings may cover some or all of the surface of the underlying core. Intermediate film layers can be provided between the core and the gelatin-containing coatings. Similarly, the gelatin-containing coatings can be provided with one or more further functional or aesthetic overcoat layers.

In one embodiment, the shell produced using the inventive gelatin formulation advantageously preferably has a high surface gloss. Surface gloss is a measure of reflected light determined according to the method set forth in an example herein. The surface gloss of the shell and/or finished dosage form is preferably at least about 150 gloss units, e.g. at least about 175 gloss units, or at least about 210 gloss units. Dosage forms with high surface gloss are preferred by consumers due to their aesthetic elegance and perceived swallowability. The surface gloss of the shell depends upon a number of factors, including the shell composition, the method of forming the shell, and, if a mold is used, the surface finish on the mold.

In one embodiment, the dosage form of this invention comprises a core having an outer surface and a shell having outer and inner surfaces, wherein the shell surrounds the core such that the shell inner surface resides substantially conformally upon the core outer surface, the shell thickness is in the range of about 100-400 microns, the relative standard deviation of the shell thickness on the dosage form is less than about 30%.

In another embodiment, the shell moisture uptake at 60 minutes of exposure to 4° C. and 75% relative humidity is less than about 0.65%.

In other embodiments the shell may contain adjuvants including plasticizers, colorants, flavors and sweeteners. Any plasticizer known in the pharmaceutical art is suitable for use in the present invention, and may include, but not be limited to polyethylene glycol; glycerin; sorbitol; triethyl citrate; triethyl amine; tribuyl citrate; dibutyl sebecate; vegetable oils such as castor oil; surfactants such as Polysorbates, sodium lauryl sulfates, and dioctyl-sodium sulfosuccinates; propylene glycol; mono acetate of glycerol; diacetate of glycerol; triacetate of glycerol; natural gums and mixtures thereof.

The amount of plasticizer in the shell is typically about 0 to about 20% by weight, preferably about 0.01 to about 5% by weight, and more preferably about 0.1 to about 3% by weight of the total weight of the shell.

Any coloring agent suitable for use in pharmaceutical application may be used in the present invention and may include, but not be limited to azo dyes, quinopthalone dyes, triphenylmethane dyes, xanthene dyes, indigoid dyes, iron oxides, iron hydroxides, titanium dioxide, natural dyes, and mixtures thereof. More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Any sweetening agent suitable for use in pharmaceutical applications may be used in the present invention and may include intense sweetener compounds such as water-soluble artificial sweeteners such as 1,2-benzisothiazol-3 (2H)-one 1, 1-dioxide (saccharin and its salts), cyclohexylsulfamic acid (cyclamate and its salts), and the potassium salt of 6-methyl-1,2,3-oxathiazin-4 (3H)-one-2,2-dioxide (Acesulfame-K, a commercially available product from Hoechst Celanese Corporation, Somerville, N.J.), proteins such as thaumatin (Talin, a commercially available product of Tate & Lyle Products, Reading, United Kingdom), chlorodeoxysugar derivatives (such as Sucralose, a commercially available product of Tate & Lyle), and dipeptides such as N-L-alpha-aspartyl-L-phenylalanine I-methyl ester (Aspartame, a commercially available product of the Nutrasweet Company, Deerfield, Ill.) and L-alpha-aspartyl-D-alanine N-(2,2,4,4-tetramethyl-3-thietanyl)amide (Alitame, a commercially available product of Pfizer, New York, N.Y.), and dihydrochalcones. The amount of sweetener in the shell is typically about 0 to about 10% by weight, preferably about 0.01 to about 3% by weight, and more preferably about 0.1 to about 2% by weight of the total weight of the shell.

In another embodiment, the dosage form is provided with at least two distinct shell portions that are compositionally different. Two shell portions are distinct from one another if separated by a continuous space such that no portion of one shell portion is in contact with another shell portion or, when provided the shell portions are provided on the dosage form, such shell portions are added in separate steps. Overlapping or abutting films strips or dipped coatings would be considered to be distinct shell portions due to the presence of a seam. In another embodiment of this invention, the shell comprises a first shell portion and a second shell portion. In one such embodiment, the first and second shell portions may comprise different shell materials having differing dissolution or erosion properties. In another such embodiment, the first and second shell materials may be visually distinct from one another, for example the visually distinct portions may be of different colors, hues, glosses, reflective qualities, brightness, depth, shades, chroma, opacity, etc. For example, the shell may have a red portion and a yellow portion, or a flat finish portion and a glossy portion, or an opaque portion and a translucent portion. In another embodiment, one or more distinct shell portions have one or more openings that expose the a portion of the core or underlying film coated core to the dissolution medium.

The following non-limiting example further illustrates the claimed invention.

### EXAMPLE 1

Part A: Preparation of Gelatin Film Solution Containing Inorganic Acid

The red gelatin solution is prepared using the composition in Table 1. 30,750 g of purified water is heated to 55° C. 17,500 g of 275 Bloom Pork Skin Gelatin is added to the water while mixing at 100 RPM. 1,000 g of Red Dye is added and allowed to mix at for 40 minutes at 55° C. until the gelatin is dissolved. 750 g of diluted phosphoric acid is added while mixing and allowed to mix an additional 5 minutes. The gelatin solution is held at 55° C. for approximately 3 hours (holding times at this temperature can generally range between about 2 and about 16 hours).

Films of this solution were cast and allowed to dry for 24 hours at ambient temperature prior to gloss measurement.

TABLE 1

| | | | Weight |
|---|---|---|---|
| Ingredient | Trade Name | Manufacturer | %* |
| DI Water | — | — | 61.5 |
| 275 Bloom | Gelatin | Gelita Corp. | 35.0 |
| Pork | | | |
| Skin Gelatin | | | |
| Phosphoric | — | Mallinckrodt-Baker Corp. | 1.5 |
| Acid Diluted | | | |
| (10%) | | | |
| Red Dye | Opatint Red DD- | Colorcon | 2.0 |
| | 1761 | | |
| TOTAL | | | 100.0 |

Part B: Surface Gloss Measurement of Film

The red gelatin film made according to Part A is tested for surface gloss using an instrument available from TriCor Systems Inc. (Elgin, Ill.) under the tradename TRI-COR MODEL 805A/806H SURFACE ANALYSIS SYSTEM and generally in accordance with the procedure described in “TriCor Systems WGLOSS 3.4 Model 805A/806H Surface Analysis System Reference Manual” (1996), which is incorporated by reference herein, except as modified below.

This instrument utilizes a CCD camera detector, employs a flat diffuse light source, compares tablet samples to a reference standard, and determines average gloss values at a 60-degree incident angle. During its operation, the instrument generates a gray-scale image, wherein the occurrence of brighter pixels indicates the presence of more gloss at that given location.

The instrument also incorporates software that utilizes a grouping method to quantify gloss, i.e., pixels with similar brightness are grouped together for averaging purposes. The “percent full scale” or “percent ideal” setting (also referred to as the “percent sample group” setting), is specified by the user to designate the portion of the brightest pixels above the threshold that will be considered as one group and averaged within that group. “Threshold,” as used herein, is defined as the maximum gloss value that will not be included in the average gloss value calculation. Thus, the background, or the non-glossy areas of a sample are excluded from the average gloss value calculations.

After initially calibrating the instrument using a calibration reference plate (190-228; 294 degree standard; no mask, rotation 0, depth 0), a standard surface gloss measurement is then created using the film prepared in Part A. The average gloss value is determined, while employing the 25 mm full view mask (190-280), and configuring the instrument to the following settings:
Rotation: 0
Depth: 0.25 inches
Gloss Threshold: 95
% Full Scale: 50%
Index of Refraction: 1.57
   The average surface gloss value for the reference standard is determined to be 269. The average surface gloss value for the sample in Part A is determined to be 286.
   Part C: Compressed Tablets

The following ingredients are mixed well in a plastic bag: 89.4 parts acetaminophen USP (590 mg/tablet) and 8.0 parts of synthetic wax X-2068 T20 (53 mg/tablet). Next, 2.1 parts of sodium starch glycolate (EXPLOTAB) (13.9 mg/tablet) and 0.09 parts of silicon dioxide (0.6 mg/tablet) are added to the bag, and mixed well. Then 0.36 parts of magnesium stearate NF (2.4 mg/tablet) are added to the bag, and the ingredients are again mixed. The resulting dry blend is compressed into tablets on a compression module as described in published U.S. Patent Application 2003/0072799 (incorporated herein by reference) using 7/16 inch extra deep concave tablet tooling. The compression module is a double row, rotary apparatus, comprising a fill zone, insertion zone, compression zone, ejection zone, and purge zone as shown in FIG. 6 of the '799 Application. The dies of the compression module are filled using vacuum assistance, with mesh screen filters located in die wall ports of each die. The resulting tablets (cores) have an average weight of 660 mg, thickness of 0.306 inches, and hardness of 3.2 kp.

Part D: Preparation of Gelatin Film Solution without Inorganic Acid

A yellow gelatin solution without inorganic acid is prepared using the composition in Table 4. 31,550 g purified water is heated to 55° C. 18,000 g of 275 Bloom Pork Skin Gelatin is added to the water while mixing at 100 RPM. 450 g of Yellow Dye is added and allowed to mix at for 40 minutes at 55° C. until the gelatin is dissolved. The gelatin solution is held at 55° C. for approximately 3 hours (holding times at this temperature can generally range between about 2 and about 16 hours).

TABLE 2

| | | | Weight |
|---|---|---|---|
| Ingredient | Trade Name | Manufacturer | %* |
| Water | — | — | 63.1 |
| 275 Bloom Pork | Gelatin | Gelita Corp. | 36.0 |
| Skin Gelatin | | | |
| Yellow Dye | Opatint Yellow | Colorcon | 0.9 |
| TOTAL | | | 100.0 |

A red gelatin solution without inorganic acid is also prepared using the composition in Table 3. 31,500 g purified water is heated to 55° C. 17,500 g of 275 Bloom Pork Skin Gelatin is added to the water while mixing at 100 RPM. 100 g of Red Dye is added and allowed to mix at for 40 minutes at 55° C. until the gelatin is dissolved. The gelatin solution is held at 55° C. for approximately 3 hours (holding times at this temperature can generally range between about 2 and about 16 hours).

TABLE 3

| | | | Weight |
|---|---|---|---|
| Ingredient | Trade Name | Manufacturer | %* |
| Water | — | — | 63.0 |
| 275 Bloom Pork | Gelatin | Gelita Corp. | 35.0 |
| Skin Gelatin | | | |
| Red Dye | Opatint Red DD- | Colorcon | 2.0 |
| | 1761 | | |
| TOTAL | | | 100.0 |

Part E: Tablet Coating

The tablets from Part C are conveyed to a thermal cycle molding module as described in published US Patent Application 2003/0086973 (incorporated herein by reference) via a transfer device as described in published US Patent Application 2003/0070903, the disclosure of which is incorporated herein by reference. The tablets are coated with red gelatin containing inorganic acid from Part A on one half thereof, and yellow gelatin on the other half thereof to form a shell. Tablets from Part C are also separately coated with the red gelatin from Table 3 without inorganic acid on one half and yellow gelatin from Table 2 without inorganic acid.

The thermal cycle molding module, which applies the shell to the tablets, is of the type shown in FIG. 28A of published US Patent Application 2003/0068367. The mold units 204 of the thermal cycle molding module comprise upper mold assemblies 214, rotatable center mold assemblies 212 and lower mold assemblies 210 as shown in FIG. 28C. Tablets are transferred to the mold assemblies, which then closed over the tablets. Shell flowable material, which is heated to a flowable state in reservoir 206, is injected into the mold cavities created by the closed mold assemblies. The temperature of the shell flowable material is then decreased, hardening it. The mold assemblies open and ejected the coated cores. Coating is performed in two steps, each half of the tablets being coated separately as shown in the flow diagram of FIG. 28B of published US Patent Application 2003/0068367 via rotation of the center mold assembly.

Part F. Dissolution Rate Results

Tablets coated with gelatin containing phosphoric acid and tablets coated with gelatin without phosphoric acid are subjected to dissolution testing after storage in open dish conditions for 12 weeks at a temperature of 40° C. and 75% relative humidity. The results are in Table 4.

All dissolutions for acetaminophen are analyzed using the following dissolution parameters: USP Type II apparatus (paddles, 50 RPM) in pH 5.8 Phosphate Buffer at 37° C. Sample aliquots of approximately 10 mL are analyzed at 15 and 30 minutes using an Agilent® UV spectrophotometer set at a wavelength of 243 nm using a 0.02 cm flow-cell.

TABLE 4

| | Avg % | Avg % |
|---|---|---|
| Sample | released in 15 min* | released in 30 min* |
| Tablets w/out | 16.5 | 79.2 |
| phosphoric acid | | |
| Tablets w/phosphoric | 63.6 | 98.0 |
| acid (1.5% in gelatin | | |
| solution) | | |

*Average % Acetaminophen released of 6 dissolution vessels

We claim:

1. A gelatin formulation comprising about 65% gelatin and at least 0.1% by weight of at least one inorganic acid, its conjugate base, associated metal salts or mixtures thereof, all percents on a dry weight basis.

2. A gelatin formulation according to claim 1 comprising about 90% gelatin on a dry weight basis.

3. A gelatin formulation according to claim 1 wherein the gelatin is mixture of derived proteins of the albuminous class that is soluble in warm water.

4. A gelatin formulation according to claim 1 wherein gelatin comprises at least about 80% 275 Bloom pork skin gelatin on a dry weight basis.

5. A gelatin formulation according to claim 1 comprising not more than 2% by dry weight of the one or more inorganic acid or its conjugate base.

6. A gelatin film coating comprising gelatin and at least 0.1% by weight of an inorganic acid or its conjugate base, wherein a free-formed surface of said gelatin film coating has a surface gloss value of at least about 150 gloss units.

7. A gelatin film coating comprising gelatin at least 0.1% by weight of an inorganic acid or its conjugate base, wherein a free-formed surface of said gelatin film coating has a surface gloss value of at least about 190 gloss units.

8. A process for the manufacturing of gelatin products with improved stability on dissolution comprising the steps of: incorporating at least one anti-crosslinking additive that consists essentially of at least one inorganic acid into gelatin before forming a final gelatin product; and forming a final gelatin product.

9. A process according to claim 8 wherein the anti-crosslinking additive is selected from the group consisting of phosphoric acid, boric acid, hydrochloric acid, sodium phosphate monobasic, potassium phosphate monobasic, sodium bisulfate and potassium bisulfate, and mixtures thereof.

10. A process according to claim 8 wherein the final gelatin product comprises at least 0.1% of an inorganic, acid on a dry weight basis as an anti-crosslinking additive.

11. A process according to claim 8 wherein the anti-crosslinking additive or the mixture of additives is incorporated in the final gelatin product in an amount of up to about 2% by dry weight.

12. A process according to claim 11, wherein the additive or the mixture of additives is incorporated in the gelatin in an amount of from about 0.1 to about 2% by weight of the dry gelatin.

13. A dosage form having a core and at least one gelatin shell portion, wherein the gelatin shell portion is a gelatin film coating according to claim 6.

14. A dosage form having a core and at least one gelatin shell portion, wherein the gelatin shell portion is a gelatin film coating according to claim 7.

15. A dosage form according to claim 13 comprising a core having an outer surface and a shell having outer and inner surfaces, wherein the shell surrounds the core such that the shell inner surface resides substantially conformally upon the core outer surface, the shell thickness is in the range of about 100-400 microns.

16. A dosage form according to claim 14 comprising a core having an outer surface and a shell having outer and inner surfaces, wherein the shell surrounds the core such that the shell inner surface resides substantially conformally upon the core outer surface, the shell thickness is in the range of about 100-400 microns.

17. A dosage form according to claim 15, wherein the relative standard deviation of the shell thickness on the dosage form is less than about 30%.

18. A dosage form according to claim 16, wherein the relative standard deviation of the shell thickness on the dosage form is less than about 30%.

19. A dosage form according to claim 13, wherein he shell moisture uptake at 60 minutes of exposure to 40° C. and 75% relative humidity is less than about 0.65%.

20. A dosage form according to claim 14, wherein he shell moisture uptake at 60 minutes of exposure to 40° C. and 75% relative humidity is less than about 0.65%.
